(19) United States
(12) Patent Application Publication
(10) Pub. No.: US 2019/0161455 A1
(43) Pub. Date: May 30, 2019

(54) METHOD FOR PRODUCING ISOCYANURIC ACID DERIVATIVE HAVING ONE HYDROCARBON GROUP

(71) Applicant: NISSAN CHEMICAL CORPORATION, Tokyo (JP)

(72) Inventors: Yuichi GOTO, Toyama-shi (JP); Gun SON, Toyama-shi (JP)

(73) Assignee: NISSAN CHEMICAL CORPORATION, Tokyo (JP)

(21) Appl. No.: 16/304,742

(22) PCT Filed: May 23, 2017
(86) PCT No.: PCT/JP2017/019247

§ 371 Date: Nov. 27, 2018

(30) Foreign Application Priority Data
May 31, 2016 (JP) 2016-109378

(51) Int. Cl. C07D 251/32

(52) U.S. Cl. CPC C07D 251/32

(57) ABSTRACT

A method for producing an isocyanuric acid derivative having one hydrocarbon group. The method having a hydrocarbon group including a first step of obtaining a compound of the following formula (1) from the compound of formula (0), a second step of obtaining a compound of the following formula (2) from the compound of formula (1), a third step of obtaining a compound of the following formula (3) from the compound of formula (2), and a fourth step of obtaining a compound of the following formula (4) from the compound of formula (3):

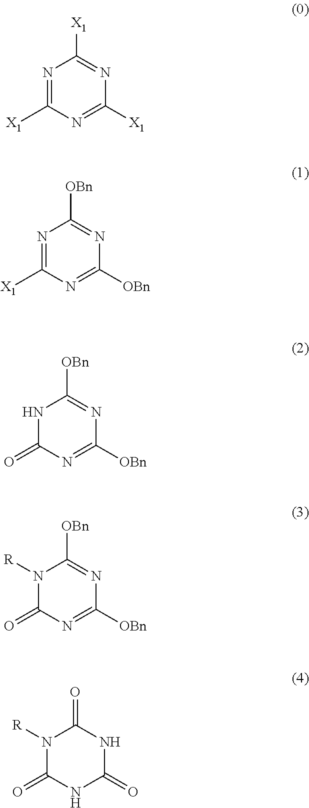

(wherein X₁s are each a chlorine atom, a fluorine atom, or a bromine atom, Bn is a benzyl group in which at least one hydrogen atom in a benzene ring is optionally substituted with methyl group, and R is a C₁₋₁₀ hydrocarbon group), wherein all the steps are carried out at a temperature of not exceeding 100° C.

### TECHNICAL FIELD

The present invention relates to a novel method for producing an isocyanuric acid derivative having a hydrocarbon group as a substituent to be bonded to a nitrogen atom.

### BACKGROUND ART

An isocyanuric acid derivative and a method for synthesizing the same have been conventionally known. For example, Non-Patent Document 1 describes a method for synthesizing a monoalkyl isocyanurate on pp. 393-396. Non-Patent Document 4 describes the content of p. 394 of Non-Patent Document 1 in detail, and on p. 3618, described that a reaction involved in synthesis of monoalkylisocyanuric acid derivative having a CH₃ group and a sec-C₄H₉ group is performed under a high-temperature condition of 250° C. Non-Patent Document 2 discloses a research result in which among 2,4,6-tris(benzyloxy)-1,3,5-triazine, 4,6-bis(benzyloxy)-1,3,5-triazine-2,4(1H,3H)-dione, and 6-(benzyloxy)-1,3,5-triazine-2,4(1H,3H)-dione, 6-(benzyloxy)-1,3,5-triazine-2,4(1H,3H)-dione is a benzylating reagent exhibiting the most excellent reactivity. Non-Patent Document 3 describes N-methylation using tetrabutylammonium isocyanurate. In the N-methylation, all mono-, di-, and tri-methyl isocyanurates are produced. Therefore, in a method described in Non-Patent Document 3, a mixture of the three types of N-methylated isocyanurates is always produced nonselectively.

An isocyanuric acid derivative is used in various applications. For example, Patent Document 1 describes an anti-reflective coating forming composition for lithography containing an isocyanuric acid derivative. Patent Document 2 describes an adhesives composition containing a polymer that is obtained by polymerizing an isocyanuric acid derivative and a monomer other than the derivative.

### PRIOR ART DOCUMENTS

### Non-Patent Documents

Non-Patent Document 1: Edwin M. Smolin; Lorence Rapoport. “Isocyanuric acid and derivatives”. The chemistry of heterocyclic compounds. s-Triazines and derivatives, INTERSCIENCE PUBLISHERS, INC., pp. 389-422 (1959)
Non-Patent Document 2: Journal of Organic Chemistry, 80, pp. 11200-11205 (2015)
Non-Patent Document 3: Tetrahedron Letters, 44, pp. 4399-4402 (2003)
Non-Patent Document 4: Journal of American Chemical Society, 75, pp. 3617-3618 (1953)

### PATENT DOCUMENTS

Patent Document 1: International publication WO 02/086624
Patent Document 2: International publication WO 2013/035787

### SUMMARY OF THE INVENTION

### Problem to be Solved by the Invention

For a conventionally known method for producing a dialkylisocyanuric acid having an alkyl group of isocyanuric acid derivatives, heating at a high temperature of 150° C. or higher for an extended period of time is required, and the selectivity of a reaction product is low. Therefore, there is a problem in which the method is not industrially useful and has a step that is concerned about safety.

### Means for Solving the Problems

The inventor of the present invention has found a method in which an isocyanuric acid derivative having a hydrocarbon group is finally produced through steps of obtaining a first intermediate from a compound of the following formula (0), for example, cyanuric chloride (also referred to as 2,4,6-trichloro-1,3,5-triazine) that is relatively cheap and easily available, as a starting material, obtaining a second intermediate, and obtaining a third intermediate. That is, an aspect of the present invention is a method for producing an isocyanuric acid derivative having a hydrocarbon group comprising a first step of obtaining a compound of the following formula (1) from the compound of formula (0), a second step of obtaining a compound of the following formula (2) from the compound of formula (1), a third step of obtaining a compound of the following formula (3) from the compound of formula (2), and a fourth step of obtaining a compound of the following formula (4) from the compound of formula (3):

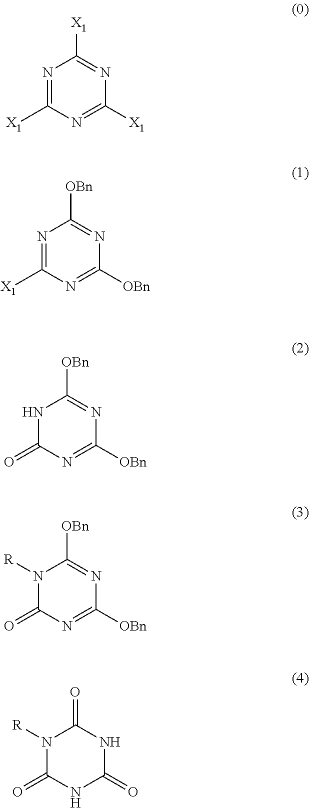

(wherein X₁s are each a chlorine atom, a fluorine atom, or a bromine atom, Bn is a benzyl group in which at least one hydrogen atom in a benzene ring is optionally substituted with methyl group, and R is a C₁₋₁₀ hydrocarbon group), wherein all the steps are carried out at a temperature of not exceeding 100° C.

For example, the C₁₋₁₀ hydrocarbon group is an alkyl group.

The first step is a process in which the compound of formula (0) is reacted with benzyl alcohol in which at least one hydrogen atom in a benzene ring is optionally substituted with methyl group in the presence of a base selected from the group consisting of a tertiary amine, an alkali metal carbonate, and an alkali metal hydrogencarbonate to obtain a reaction product, and the reaction product is washed with alcohols to obtain the compound of formula (1). The benzyl alcohol in which at least one hydrogen atom in the benzene ring is optionally substituted with methyl group and the base can be each used in an amount of 2.7 molar equivalents to 5.0 molar equivalents relative to 1.0 molar equivalent of the compound of formula (0).

The base used in the first step may be a tertiary amine. Examples of the tertiary amine include diisopropylethylamine. As the base, an alkali metal carbonate or an alkali metal hydrogencarbonate may be used instead of the tertiary amine. Examples of the alkali metal carbonate include sodium carbonate. Examples of the alkali metal hydrogencarbonate include sodium hydrogencarbonate. Examples of the benzyl alcohol in which at least one hydrogen atom in the benzene ring is optionally substituted with methyl group include benzyl alcohol and 4-methylbenzenemethanol.

Examples of the alcohol used for washing the reaction product in the first step include ethanol.

The second step is a process in which the compound of formula (1) is reacted in a solution containing a heterocyclic compound selected from the group consisting of N-methylmorpholine, N-ethylmorpholine, N-methylpiperidine, N-ethylpiperidine, N-methylpyrrolidine, and N-ethylpyrrolidine and acetic acid or formic acid to obtain a reaction product, and the reaction product is washed with at least one solvent selected from the group consisting of esters, aromatic hydrocarbons, alcohols, and ethers to obtain the compound of formula (2). The heterocyclic compound has at least one nitrogen atom as a heteroatom, and a substituent at an N position.

Examples of the at least one solvent used for washing the reaction product in the second step may be an aromatic hydrocarbons. Examples of the aromatic hydrocarbons include toluene. The at least one solvent may be esters, alcohols, or ethers. Examples of the esters include ethyl acetate. Examples of the alcohols include ethanol. Examples of the ethers include cyclopentyl methyl ether.

The third step is a process in which the compound of formula (2) is reacted with a compound of the following formula (5), (6), (7), or (8) having a C₁₋₁₀ hydrocarbon group in the presence of alkali metal carbonate to obtain a reaction product, and the reaction product is washed with at least one solvent selected from the group consisting of alcohols and ethers to obtain the compound of formula (3).

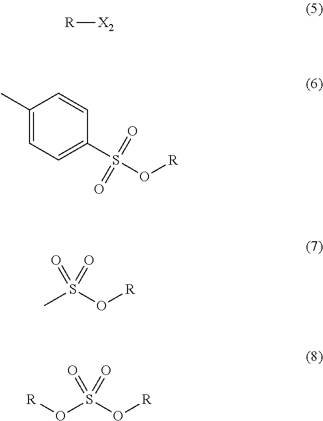

(wherein R is a C₁₋₁₀ hydrocarbon group, and X₂ is a halogen atom.)

The compound of formula (5), (6), (7), or (8) used in the third step is, for example, an alkylating agent selected from the group consisting of halogenated alkyl, alkyl tosylate, alkyl mesylate, and dialkyl sulfate.

The alkylating agent may be halogenated alkyl or dialkyl sulfate. Examples of the halogenated alkyl include methyl iodide, ethyl bromide, propyl bromide, allyl bromide, and propargyl bromide. Examples of the dialkyl sulfate include dimethyl sulfate. As the alkylating agent, alkyl tosylate or alkyl mesylate may be used instead of halogenated alkyl and dialkyl sulfate. Examples of the alkyl tosylate include methyl p-toluenesulfonate and ethyl p-toluenesulfonate. Examples of the alkyl mesylate include ethyl methanesufonate.

Examples of the alkali metal carbonate used in the third step include potassium carbonate and cesium carbonate.

The at least one solvent used for washing the reaction product in the third step may be alcohols. Examples of the alcohols include ethanol. The at least one solvent may be ethers. Examples of the ethers include cyclopentyl methyl ether.

The fourth step is a process in which the compound of formula (3) is reacted with an alcohol compound in the presence of trifluoromethanesulfonic acid or trimethylsilyl trifluoromethanesulfonate to obtain a reaction product, and the reaction product is washed with at least one solvent selected from the group consisting of esters, halogenated alkyls, and alcohols to obtain the compound of formula (4).

Examples of the alcohol compound used in the fourth step include methanol.

The at least one solvent used for washing the reaction product in the fourth step may be esters. Examples of the esters include ethyl acetate. The at least one solvent may be also halogenated alkyls or alcohols. Examples of the halogenated alkyls include chloroform. Examples of the alcohols include ethanol.

The first to fourth steps are carried out at a temperature of not exceeding 100° C. The temperature of not exceeding 100° C. is between 0° C. and 100° C., for example, 0° C. to 50° C.

### Effects of the Invention

A method for producing an isocyanuric acid derivative having a hydrocarbon group according to the present invention does not include a step carried out at a temperature of higher than 100° C. among all the steps, and does not require purification through column chromatography during purification of reaction products. Therefore, this method is industrially useful. According to the present invention, the isocyanuric acid derivative having a hydrocarbon group that is a target can be obtained with a high purity (purity: 98% or more) as compared with a conventional method.

### BRIEF DESCRIPTION OF THE DRAWINGS

FIG. 1 is a chromatogram illustrating a result of measurement of a compound of Example 1 obtained in a first step by high performance chromatography.

FIG. 2 is a chromatogram illustrating a result of measurement of a compound of Example 4 obtained in a second step by high performance liquid chromatography.

FIG. 3 is a chromatogram illustrating a result of measurement of a compound of Example 7 obtained in a third step by high performance liquid chromatography.

FIG. 4 is a chromatogram illustrating a result of measurement of a compound of Example 9 obtained in a fourth step by high performance liquid chromatography.

FIG. 5 is a chromatogram illustrating a result of measurement of a compound obtained in Comparative Example 16 by high performance liquid chromatography.

### MODES FOR CARRYING OUT THE INVENTION

The method for producing an isocyanuric acid derivative having a hydrocarbon group according to the present invention includes the first to fourth steps. Therefore, the compound of formula (4) in which a hydrocarbon group is introduced as a substituent bonded to a nitrogen atom in isocyanuric acid is obtained. In formula (4), R is a C₁₋₁₀ hydrocarbon group introduced. The hydrocarbon group may be a linear, branched, or cyclic hydrocarbon group, and have at least one double bond or triple bond. When the hydrocarbon group is an alkyl group, examples of the alkyl group include methyl group, ethyl group, n-propyl group, isopropyl group, n-butyl group, isobutyl group, n-pentyl group, n-hexyl group, n-heptyl group, n-octyl group, n-pentyl group, n-nonyl group, n-decyl group, cyclohexylmethyl group, and cyclopentylmethyl group. Examples of the hydrocarbon group except for the alkyl group include benzyl group, allyl group, and propargyl group.

In the first step of the production method of the present invention, the compound of formula (0) is reacted with benzyl alcohol in which at least one hydrogen atom in a benzene ring may be substituted with methyl group in the presence of a base such as a tertiary amine. By this reaction, the compound of formula (1) in which two substituents X₁s among three substituents X₁s in the compound of formula (0) are substituted with benzyloxy group derived from the benzyl alcohol can be obtained. For example, when as the compound of formula (0), cyanuric chloride is used, the compound of formula (1) (wherein X₁ is a chlorine atom) in which two chlorine atoms among three chlorine atoms in cyanuric chloride are substituted with benzyloxy group can be obtained by the aforementioned reaction. In order to preferentially obtain the compound of formula (1), the benzyl alcohol and the base such as a tertiary amine are each used preferably in an amount of 2.7 molar equivalents to 5.0 molar equivalents, and more preferably in an amount of 4.0 molar equivalents to 5.0 molar equivalents, relative to 1.0 molar equivalent of the compound of formula (0) such as cyanuric chloride. When the benzyl alcohol and the base such as a tertiary amine are each used in an amount of less than 2.0 molar equivalents relative to 1.0 molar equivalent of the compound of formula (0), a compound in which one substituent X₁ among three substituents X₁s in the compound of formula (0) is substituted with benzyloxy group derived from the benzyl alcohol is preferentially by-produced as compared with the compound of formula (1) that is a target. Therefore, the yield of the compound of formula (1) is decreased.

Preferable examples of the tertiary amine used in the aforementioned reaction include diisopropylethylamine, triethylamine, tributylamine, and 1,8-diazabicyclo[5.4.0]-7-undecene. When the reaction is carried out in a solution, a solvent is preferably halogenated alkyls. Examples of the halogenated alkyls include chloroform, dichloromethane, carbon tetrachloride, and dichloroethane.

Specifically, the aforementioned reaction is started by adding dropwise a mixed solution of the benzyl alcohol in which at least one hydrogen atom in a benzene ring may be substituted with methyl group, the base such as a tertiary amine, and the halogenated alkyls to a mixed solution of the compound of formula (0) such as cyanuric chloride, and the halogenated alkyls. The temperature during the dropwise addition is preferably 0° C. to 5° C. When the addition dropwise is carried out at a temperature of higher than 5° C., a by-product is produced with the compound of formula (1) that is a target. Therefore, the purity and yield of the compound of formula (1) are decreased. The reaction temperature after the dropwise addition is not particularly limited, and is usually 0° C. to 40° C., and preferably 20° C. to 30° C. The reaction time is usually 12 hours to 24 hours, and preferably 15 hours to 18 hours.

A reaction product obtained by the aforementioned reaction is subjected to a separation operation, resulting in concentration. The concentrated reaction product is then washed with alcohols to obtain the compound of formula (1). Preferable examples of the alcohols used in washing include ethanol, methanol, isopropanol, n-propanol, sec-butyl alcohol, tert-butyl alcohol, n-butanol, and cyclohexanol. The amount of a solvent used in the washing is preferably 0.5 to 3.0 times, and more preferably 2.0 times the mass of the compound of formula (0) (e.g., cyanuric chloride). The temperature during the washing is not particularly limited, and is usually 0° C. to 40° C., and preferably 0° C. to 5° C. The washing time is usually 10 minutes to 1 hour, and preferably 30 minutes to 1 hour.

In the second step of the production method of the present invention, the compound of formula (1) is reacted in the presence of a heterocyclic compound selected from the group consisting of N-methylmorpholine, N-ethylmorpholine, N-methylpiperidine, N-ethylpiperidine, N-methylpyrrolidine, and N-ethylpyrrolidine, and acetic acid or formic acid. When the reaction is carried out in a solution, a solvent is preferably alcohols. Examples of the alcohols include methanol, ethanol, isopropanol, n-propanol, sec-butyl alcohol, tert-butyl alcohol, n-butanol, and cyclohexanol.

Specifically, the aforementioned reaction is started by adding the compound of formula (1) to a mixed solution of the heterocyclic compound selected from the group consisting of N-methylmorpholine, N-ethylmorpholine, N-methylpiperidine, N-ethylpiperidine, N-methylpyrrolidine, and N-ethylpyrrolidine, acetic acid or formic acid, and the alcohols. The temperature during the addition is preferably 0° C. to 5° C. The reaction temperature after the addition is not particularly limited, and is usually 0° C. to 40° C., and preferably 20° C. to 30° C. The reaction time is usually 30 minutes to 3 hours, and preferably 1 hour to 2 hours.

A reaction product obtained by the aforementioned reaction is subjected to a separation operation, resulting in concentration. The concentrated reaction product is then washed with at least one solvent selected from the group consisting of esters, aromatic hydrocarbons, alcohols, and ethers to obtain the compound of formula (2). When the solvent used in the washing is esters, preferable examples thereof include ethyl acetate, methyl acetate, butyl acetate, and methyl propionate. When the solvent is an aromatic hydrocarbon, preferable examples thereof include toluene, benzene, xylene, mesitylene, chlorobenzene, dichlorobenzene, nitrobenzene, and tetralin. When the solvent is alcohols, preferable examples thereof include ethanol, methanol, isopropanol, n-propanol, sec-butyl alcohol, tert-butyl alcohol, n-butanol, and cyclohexanol. When the solvent is ethers, preferable examples thereof include cyclopentyl methyl ether, diethyl ether, diisopropyl ether, methyl tert-butyl ether, tetrahydrofuran, and dioxane. The amount of the solvent used in the washing is preferably 0.5 to 3.0 times, and more preferably 2.0 times the mass of the compound of formula (1). The temperature during the washing is not particularly limited, and is usually 0° C. to 40° C., and preferably 20° C. to 30° C. The washing time is usually 10 minutes to 1 hour, and preferably 10 minutes to 30 minutes.

In the third step of the production method of the present invention, the compound of formula (2) is reacted with the compound of formula (5), (6), (7), or (8) in the presence of an alkali metal carbonate. The amount of the compound of formula (5), (6), (7), or (8) used is preferably 1.0 molar equivalent to 1.5 molar equivalents, and more preferably 1.25 molar equivalents, relative to 1.0 molar equivalent of the compound of formula (2). The amount of the alkali metal carbonate used is preferably 1.0 molar equivalent to 1.5 molar equivalents, and more preferably 1.25 molar equivalents, relative to 1.0 molar equivalent of the compound of formula (2). The temperature during the reaction is not particularly limited, and is usually 0° C. to 40° C., and preferably 20° C. to 30° C. The reaction time is usually 30 minutes to 2 hours, and preferably 30 minutes to 1 hour. When the aforementioned reaction is carried out in a solution, it is preferable that as a solvent, at least one solvent selected from the group consisting of aprotic polar solvents and aromatic hydrocarbons be used. Examples of the aprotic polar solvents include dimethylsulfoxide, N-methylpyrrolidone, dimethylacetamide, and dimethylfonnamide. When the solvent is aromatic hydrocarbons, preferable examples thereof include toluene, benzene, xylene, mesitylene, chlorobenzene, dichlorobenzene, nitrobenzene, and tetralin.

When the compound of formula (5), (6), (7), or (8) used in the third step of the production method of the present invention is selected, the type of hydrocarbon group introduced as a substituent to be bonded to a nitrogen atom in isocyanuric acid is determined. For example, as the compound of formula (5), (6), (7), or (8), an alkylating agent having a methyl group is used to introduce the methyl group, or an alkylating agent having an ethyl group is used to introduce the ethyl group.

A reaction product obtained by the aforementioned reaction is subjected to a separation operation, resulting in concentration. The concentrated reaction product is then washed with at least one solvent selected from the group consisting of alcohols and ethers, to obtain the compound of formula (3). When the solvent used in the washing is alcohols, preferable examples thereof include ethanol, methanol, isopropanol, n-propanol, -butyl alcohol, tert-butyl alcohol, n-butanol, and cyclohexanol. When the solvent is ethers, preferable examples thereof include cyclopentyl methyl ether, diethyl ether, diisopropyl ether, methyl tert-butyl ether, tetrahydrofuran, and dioxane. The amount of the solvent used in the washing is preferably 0.5 to 3.0 times, and more preferably 2.0 times the mass of the compound of formula (1). The temperature in the washing is not particularly limited, and is usually 0° C. to 40° C., and preferably 0° C. to 5° C. The washing time is usually 10 minutes to 1 hour, and preferably 10 minutes to 30 minutes.

In the fourth step of the production method of the present invention, the compound of formula (3) is reacted with an alcohol compound in the presence of trifluoromethanesulfonic acid or trimethylsilyl trifluoromethanesulfonate. Examples of the alcohol compound include methanol, ethanol, isopropanol, n-propanol, sec-butyl alcohol, tert-butyl alcohol, n-butanol, cyclohexanol, and phenol. The amount of the alcohol compound used is preferably 2 molar equivalents to 3 molar equivalents, and more preferably 2.4 molar equivalents, relative to 1.0 molar equivalent of the compound of formula (3). The amount of the trifluoromethanesulfonic acid or trimethylsilyl trifluoromethanesulfonate used is preferably 0.7 molar equivalents to 1.0 molar equivalent, and more preferably 1.0 molar equivalent, relative to 1.0 molar equivalent of the compound of formula (3). When the amount is less than 0.7 molar equivalents, an impurity different from the compound of formula (4) that is a target is produced as a by-product. Therefore, the purity and yield of the compound of formula (4) are decreased. The temperature during the reaction is not particularly limited, and is usually 0° C. to 40° C., and preferably 20° C. to 30° C. The reaction time is usually 1 hour to 5 hours, and preferably 1 hour to 2 hours. When the aforementioned reaction is carried out in a solution, a solvent is preferably ethers and aromatic hydrocarbons. Examples of the ethers include diethyl ether, diisopropyl ether, methyl tert-butyl ether, cyclopentyl methyl ether, tetrahydrofuran, and dioxane. Preferable examples of the aromatic hydrocarbons include toluene, benzene, xylene, mesitylene, chlorobenzene, dichlorobenzene, nitrobenzene, and tetralin.

To a reaction product obtained by the aforementioned reaction, an organic base is added. Concentration is carried out. The concentrated reaction product is washed with at least one solvent selected from the group consisting of esters, halogenated alkyls, and alcohols. As a result, the compound of formula (4) is obtained. Preferable examples of the organic base include pyridine, 4-dimethylaminopyridine, triethylamine, tributylamine, N,N-dimethylaniline, and 1,8-diazabicyclo[5.4.0]-7-undecene. The amount of the organic base used is preferably 1.0 molar equivalent to 2.0 molar equivalents, and more preferably 1.0 molar equivalent, relative to 1.0 molar equivalent of the compound of formula (3). When the solvent used in the washing is esters, preferable examples thereof include ethyl acetate, methyl acetate, butyl acetate, and methyl propionate. When the solvent is halogenated alkyls, preferable examples thereof include chloroform, dichloromethane, carbon tetrachloride, and dichloroethane. When the solvent is alcohols, preferable examples thereof include ethanol, methanol, isopropanol, n-propanol, sec-butyl alcohol, tert-butyl alcohol, n-butanol, and cyclohexanol. The amount of the solvent used in the washing is preferably 2.0 to 5.0 times, and more preferably 3.0 times the mass of the compound of formula (3). The temperature during the washing is not particularly limited, and is usually 0° C. to 40° C., and preferably 20° C. to 30° C. The washing time is usually 10 minutes to 1 hour, and preferably 10 minutes to 30 minutes.

### EXAMPLES

Hereinafter, the method for producing an isocyanuric acid derivative having a hydrocarbon group according to the present invention will be described with reference to specific examples. However, the present invention is not limited to the specific examples described below.

[HPLC Analysis Condition-1]

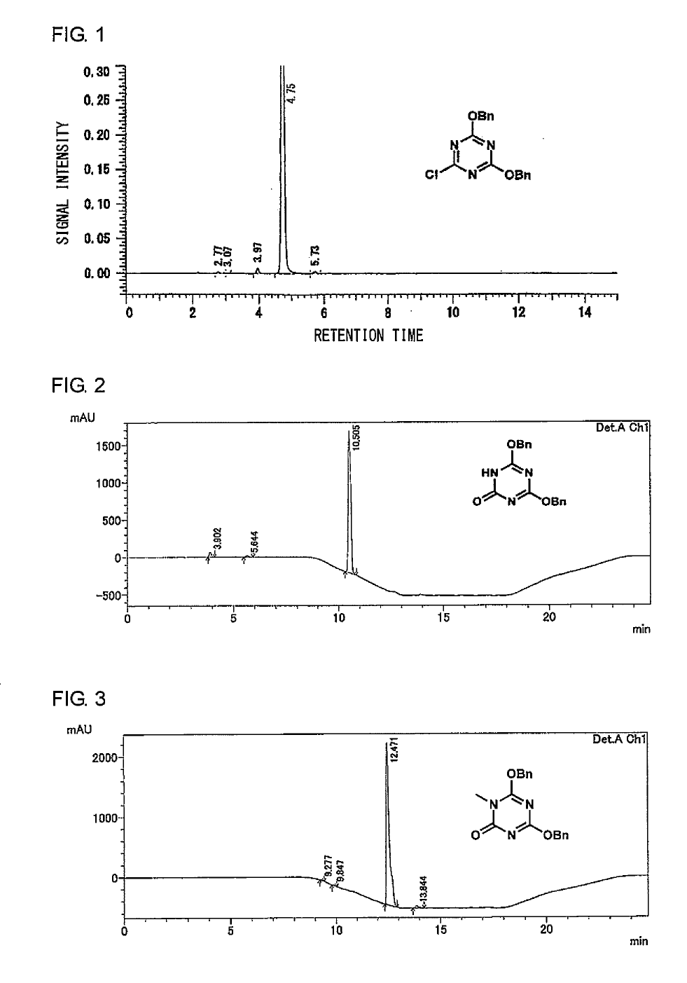

Purities shown in examples described below are results of measurement by high performance liquid chromatography (hereinafter abbreviated as HPLC). A measurement condition and the like are as follows. The purities are each obtained by dividing a peak of each component as shown in FIG. 1, and calculating the area value of each peak of the respective components in percentage.

Apparatus: L2000 series manufactured by Hitachi High-Technologies Corporation
Column: XBridge [registered trademark] BEH C18 Column, 130 Å, 5 μm, 4.6 mm×250 mm (manufactured by Nihon Waters K.K.)
Eluent: acetonitrile/0.2% ammonium acetate aqueous solution=8/2 (v/v)
Flow rate: 1.0 mL/min

### Detector: UV (254 nm)

Column temperature: 40° C.
Analysis time: 15 minutes
Injection volume: 2.0 μL
Diluted solvent: acetonitrile

[HPLC Analysis Condition-2]

The purities shown in examples described below are results of measurement by HPLC. A measurement condition and the like are as follows. The purities are each obtained by dividing a peak of each component as shown in FIGS. 2, 3, and 4, and calculating the area value of each peak in percentage.

Apparatus: LC-2010A manufactured by Shimadzu Corporation
Column: XBridge [registered trademark] BEH C18 Column, 130 Å, 5 μm, 4.6 mm×250 mm (manufactured by Nihon Waters K.K.)
Eluent: acetonitrile/0.2% ammonium acetate aqueous solution=3/7 (v/v) (0 minute to 5 minutes), the composite ratio was changed from 3/7 (v/v) to 8/2 (v/v) (5 minutes to 10 minutes), 8/2 (v/v) (10 minutes to 15 minutes)
Flow rate: 1.0 mL/min

### Detector: UV (210 nm)

Column temperature: 40° C.
Analysis time: 25 minutes
Injection volume: 1.0 μL
Diluted solvent: acetonitrile/water=1/1 (w/w)

[Method for Calculating Yield]

Yields shown in examples described below are each calculated from the weight and theoretical yield of a compound to be obtained in percentage. The theoretical yield is obtained by multiplying the number of moles of a raw material compound used in synthesis by the molecular weight of the compound to be obtained.

[First Step]

### Synthesis Example 1

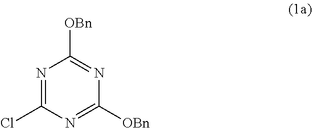 [SMILES: Clc1nc(OCc2ccccc2)nc(OCc2ccccc2)n1]

20.00 g of cyanuric chloride (available from Tokyo Chemical Industry Co., Ltd.) and 120.00 g of chloroform were mixed, and the mixture was cooled to 0° C. with stirring. To the mixture, a mixed solution of 46.91 g of benzyl alcohol (available from Kanto Chemical Co., Inc.), 56.07 g of diisopropylethylamine, and 60.00 g of chloroform was added dropwise. After completion of dropwise addition, the temperature was increased to 25° C., and the mixture was stirred for 17 hours to obtain a reaction solution. To the reaction solution, 200.00 g of saturated NH₄Cl aqueous solution was added, and separation was carried out. To the separated organic phase, 200.00 g of saturated saline solution was then added, and separation was carried out. This operation was repeated twice. From the organic phase, the solvent was distilled off under reduced pressure, and the residue was then dried at 40° C. under reduced pressure. As a result, 57.56 g of a triazine compound of formula (1a) described above was obtained as a crude (crude product) (yield of the crude: >99.9%). The obtained compound was subjected to measurement under the HPLC analysis condition-1. The purity thereof was 61.4%.

### Example 1

To 3.00 of the crude of the triazine compound obtained in Synthesis Example 1, 2.08 g of ethanol was added, and the mixture was stirred at 25° C. for 10 minutes. After stirring, the mixture was subjected to filtration, and the cake was washed with 1.04 g of ethanol twice. Herein, the cake is a solid material that remains after a liquid is separated from a solid-liquid mixture such as a slurry by filtration. The resulting crystal was dried at 40° C. under reduced pressure to obtain 1.30 g of the triazine compound of formula (1a) as a pale yellow solid (yield: 70.4%). The obtained compound was subjected to measurement under the HPLC analysis condition-1. The purity thereof was 98.4%. A chromatogram obtained by the measurement is shown in FIG. 1.

### Example 2

1.49 g of the triazine compound of formula (1a) was obtained as a pale yellow solid (yield: 80.4%) in the same manner as in Example 1 except that 2.08 g of ethanol was added to 3.00 g of the crude of the triazine compound obtained in Synthesis Example 1 and the mixture was stirred at 0° C. for 10 minutes. The obtained compound was subjected to measurement under the HPLC analysis condition-1. The purity thereof was 98.2%.

### Comparative Example 1

The same operation as in Example 1 except that 2.08 g of toluene was added to 3.00 g of the crude of the triazine compound obtained in Synthesis Example 1 was carried out. After stirring, the compound was completely dissolved. As a result, a crystal was not obtained.

### Comparative Example 2

1.63 g of the triazine compound of formula (1a) was obtained as a pale yellow solid (yield: 87.9%) in the same manner as in Example 1 except that 2.08 g of heptane was added to 3.00 g of the crude of the triazine compound obtained in Synthesis Example 1 and heptane was used for a solvent for washing the cake. The obtained compound was subjected to measurement under the HPLC analysis condition-1. The purity thereof was 76.8%.

### Comparative Example 3

1.10 g of the triazine compound of formula (1a) was obtained as a pale yellow solid (yield: 59.6%) in the same manner as in Example 1 except that 2.08 g of ethyl acetate/heptane at a ratio of 1/9 (w/w) was added to 3.00 g of the crude of the triazine compound obtained in Synthesis Example 1 and ethyl acetate/heptane at a ratio of 1/9 (w/w) was used for a solvent for washing the cake. The obtained compound was subjected to measurement under the HPLC analysis condition-1. The purity thereof was 86.1%.

### Comparative Example 4

3.00 g of the crude of the triazine compound obtained in Synthesis Example 1 was subjected to silica gel chromatography using ethyl acetate/hexane at a ratio of 1/10 (w/w) as a developing solvent. The obtained solution was concentrated at 40° C. and dried under reduced pressure to obtain 1.58 g of the triazine compound of formula (1a) as a white solid (yield: 85.3%). The obtained compound was subjected to measurement under the HPLC analysis condition-1. The purity thereof was 92.5%.

TABLE 1

| | | Yield [%] | Purity [%] |
|---|---|---|---|
| Example 1 | Ethanol | 70.4 | 98.4 |
| Example 2 | Ethanol (0° C.) | 80.4 | 98.2 |
| Comparative | Toluene | Dissolved | |
| Example 1 | | | |
| Comparative | Heptane | 87.9 | 76.8 |
| Example 2 | | | |
| Comparative | Ethyl acetate/heptane = | 59.6 | 86.1 |
| Example 3 | 1/9(w/w) | | |
| Comparative | Column | 85.3 | 92.5 |
| Example 4 | | | |

[Second Step]

### Synthesis Example 2

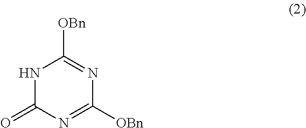 [SMILES: O=c1nc(OCc2ccccc2)nc(OCc2ccccc2)[nH]1]

30.86 g of N-methylmorpholine (available from Tokyo Chemical Industry Co., Ltd.), 9.16 g of acetic acid (available from Kanto Chemical Co., Inc.), and 250.00 g of methanol were mixed, and the mixture was cooled to 0° C. To the mixture, 25.00 g of the triazine compound obtained in Example 1 was added with stirring. Subsequently, the temperature was increased to 25° C., and the mixture was stirred for 1 hour to obtain a reaction solution. To the reaction solution, 9.16 g of acetic acid was added, and the solvent was distilled off under reduced pressure. To the residue, 250.00 g of chloroform and 250.00 g of 1M HCl were added, and separation was carried out. To the separated organic phase, 250.00 g of saturated saline solution was added, and separation was carried out. The separated organic phase was dried at 40° C. under reduced pressure to obtain 22.98 g of triazin-one compound of formula (2) described above as a crude (crude product) (yield of the crude: 97.4%). The obtained compound was subjected to measurement under the HPLC analysis condition-2. The purity thereof was 88.7%.

### Example 3

To 3.00 of the crude of the triazin-one compound obtained in Synthesis Example 2, 6.53 g of ethyl acetate was added, and the mixture was stirred at 25° C. for 10 minutes. After stirring, the mixture was subjected to filtration, and the cake was washed with 3.26 g of ethyl acetate twice. The resulting crystal was dried at 40° C. under reduced pressure, to obtain 2.56 g of the triazin-one compound of formula (2) as a white solid (yield: 83.3%). The obtained compound was subjected to measurement under the HPLC analysis condition-2. The purity thereof was 95.8%.

### Example 4

2.66 g of the triazin-one compound of formula (2) was obtained as a white solid (yield: 86.4%) in the same manner as in Example 3 except that 6.53 g of toluene was added to 3.00 g of the crude of the triazin-one compound obtained in Synthesis Example 2 and toluene was used for a solvent for washing the cake. The obtained compound was subjected to measurement under the HPLC analysis condition 2. The purity thereof was 96.0%. A chromatogram obtained by the measurement is shown in FIG. 2.

### Example 5

2.54 g of the triazin-one compound of formula (2) was obtained as a white solid (yield: 82.4%) in the same manner as in Example 3 except that 6.53 g of ethanol was added to 3.00 g of the crude of the triazin-one compound obtained in Synthesis Example 2 and ethanol was used for a solvent for washing the cake. The obtained compound was subjected to measurement under the HPLC analysis condition-2. The purity thereof was 96.1%.

### Example 6

2.64 g of the triazin-one compound of formula (2) was obtained as a white solid (yield: 85.8%) in the same manner as in Example 3 except that 6.53 g of cyclopentyl methyl ether was added to 3.00 g of the crude of the triazin-one compound obtained in Synthesis Example 2 and cyclopentyl methyl ether was used for a solvent for washing the cake. The obtained compound was subjected to measurement under the HPLC analysis condition-2. The purity thereof was 95.9%.

### Comparative Example 5

2.73 g of the triazin-one compound of formula (2) was obtained as a white solid (yield: 88.7%) in the same manner as in Example 3 except that 6.53 g of heptane was added to 3.00 g of the crude of the triazin-one compound obtained in Synthesis Example 2 and heptane was used for a solvent for washing the cake. The obtained compound was subjected to measurement under the HPLC analysis condition-2. The purity thereof was 92.8%.

### Comparative Example 6

1.88 g of the triazin-one compound of formula (2) was obtained as a white solid (yield: 61.0%) in the same manner as in Example 3 except that 6.53 g of chloroform was added to 3.00 g of the crude of the triazin-one compound obtained in Synthesis Example 2 and chloroform was used for a solvent for washing the cake. The obtained compound was subjected to measurement under the HPLC analysis condition-2. The purity thereof was 96.6%.

### Comparative Example 7

3.00 g of the crude of the triazin-one compound obtained in Synthesis Example 2 was subjected to silica gel chromatography using chloroform/ethyl acetate=at a ratio of 4/1 (w/w) as a developing solvent. The obtained solution was concentrated at 40° C. and dried under reduced pressure to obtain 2.42 g of the triazin-one compound of formula (2) as a white solid (yield: 78.6%). The obtained compound was subjected to measurement under the HPLC analysis condition-1. The purity thereof was 99.7%.

TABLE 2

| | | Yield [%] | Purity [%] |
|---|---|---|---|
| Example 3 | Ethyl acetate | 83.3 | 95.8 |
| Example 4 | Toluene | 86.4 | 96.0 |
| Example 5 | Ethanol | 82.4 | 96.1 |
| Example 6 | Cyclopentyl methyl ether | 85.8 | 95.9 |
| Comparative | Heptane | 88.7 | 92.8 |
| Example 5 | | | |
| Comparative | Chloroform | 61.0 | 96.6 |
| Example 6 | | | |
| Comparative | Column | 78.6 | 99.7 |
| Example 7 | | | |

[Third Step]

### Synthesis Example 3

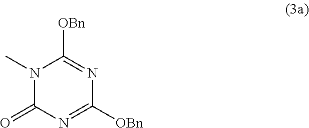 [SMILES: Cn1c(OCc2ccccc2)nc(OCc2ccccc2)nc1=O]

21.00 g of the triazin-one compound obtained in Example 4, 27.65 g of cesium carbonate (available from Tokyo Chemical Industry Co., Ltd.), and 210.00 g of dimethyl sulfoxide were mixed, and the mixture was stirred at 25° C. To the mixture, 12.05 g of iodomethane (available from Tokyo Chemical Industry Co., Ltd.) was added dropwise. After completion of dropwise addition, the mixture was stirred at 25° C. for 1 hour to obtain a reaction solution. To the reaction solution, 210.00 g of toluene was added, and the mixture was subjected to filtration. To the filtrate, 210.00 g of water was then added, and separation was carried out. This operation was repeated twice. From the organic phase, the solvent was distilled off under reduced pressure, and the residue was then dried at 40° C. under reduced pressure. As a result, 20.89 g of monomethyl triazin-one compound of formula (3a) described above was obtained as a crude (crude product) (yield of the crude: 95.2%). The obtained compound was subjected to measurement under the HPLC analysis condition-2. The purity thereof was 85.6%.

### Example 7

To 3.00 of the crude of the monomethyl triazin-one compound obtained in Synthesis Example 3, 6.03 g of ethanol was added, and the mixture was stirred at 25° C. for 10 minutes. After stirring, the mixture was subjected to filtration, and the cake was washed with 3.02 g of ethanol twice. The resulting crystal was dried at 40° C. under reduced pressure to obtain 2.30 g of the monomethyl triazin-one compound of formula (3a) as a white solid (yield: 73.0%). The obtained compound was subjected to measurement under the HPLC analysis condition-2. The purity thereof was 98.0%. This compound was subjected to ¹H NMR measurement (500 MHz, CDCl₃). A measurement result of ¹H NMR: δ 7.48-7.34 (m, 10H), 5.48 (s, 2H), 5.44 (s, 2H), 3.41 (s, 3H). A chromatogram obtained by the measurement is shown in FIG. 3.

### Example 8

2.30 g of the monomethyl triazin-one compound of formula (3a) was obtained as a white solid (yield: 72.9%) in the same manner as in Example 7 except that 6.03 g of cyclopentyl methyl ether was added to 3.00 g of the crude of the monomethyl triazin-one compound obtained in Synthesis Example 3 and cyclopentyl methyl ether was used for a solvent for washing the cake. The obtained compound was subjected to measurement under the HPLC analysis condition-2. The purity thereof was 98.5%.

### Comparative Example 8

2.77 g of the monomethyl triazin-one compound of formula (3a) was obtained as a white solid (yield: 87.8%) in the same manner as in Example 7 except that 6.03 g of heptane was added to 3.00 g of the crude of the monomethyl triazin-one compound obtained in Synthesis Example 3 and heptane was used for a solvent for washing the cake. The obtained compound was subjected to measurement under the HPLC analysis condition-2. The purity thereof was 89.8%.

### Comparative Example 9

1.69 g of the monomethyl triazin-one compound of formula (3a) was obtained as a white solid (yield: 53.6%) in the same manner as in Example 7 except that 6.03 g of ethyl acetate was added to 3.00 g of the crude of the monomethyl triazin-one compound obtained in Synthesis Example 3 and ethyl acetate was used for a solvent for washing the cake. The obtained compound was subjected to measurement under the HPLC analysis condition-2. The purity thereof was 98.7%.

### Comparative Example 10

1.57 g of the monomethyl triazin-one compound of formula (3a) was obtained as a white solid (yield: 49.8%) in the same manner as in Example 7 except that 6.03 g of toluene was added to 3.00 g of the crude of the monomethyl triazin-one compound obtained in Synthesis Example 3 and toluene was used for a solvent for washing the cake. The obtained compound was subjected to measurement under the HPLC analysis condition-2. The purity thereof was 98.4%.

### Comparative Example 11

The same operation as in Example 7 except that 6.03 g of chloroform was added to 3.00 g of the crude of the monomethyl triazin-one compound obtained in Synthesis Example 3 was carried out. After stirring, the compound was completely dissolved. As a result, a crystal was not obtained.

### Comparative Example 12

3.00 g of the crude of the monomethyl triazin-one compound obtained in Synthesis Example 3 was subjected to silica gel chromatography using chloroform and heptane at a ratio of 9/1 (w/w) instead of chloroform and heptane at a ratio of 7/3 (w/w), and further chloroform and ethyl acetate at a ratio 9/1 (w/w) as a developing solvent. The obtained solution was concentrated at 40° C. and dried under reduced pressure, to obtain 2.82 g of the triazin-one compound of formula (3a) as a white solid (yield: 89.5%). The obtained compound was subjected to measurement under the HPLC analysis condition-2. The purity thereof was 91.4%.

TABLE 3

| | | Yield [%] | Purity [%] |
|---|---|---|---|
| Example 7 | Ethanol | 73.0 | 98.0 |
| Example 8 | Cyclopentyl methyl ether | 72.9 | 98.5 |
| Comparative | Heptane | 87.8 | 89.8 |
| Example 8 | | | |
| Comparative | Ethyl acetate | 53.6 | 98.7 |
| Example 9 | | | |
| Comparative | Toluene | 49.8 | 98.4 |
| Example 10 | | | |
| Comparative | Chloroform | Dissolved | |
| Example 11 | | | |
| Comparative | Column | 89.5 | 91.4 |
| Example 12 | | | |

[Fourth Step]

### Example 9

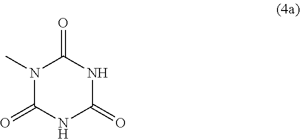

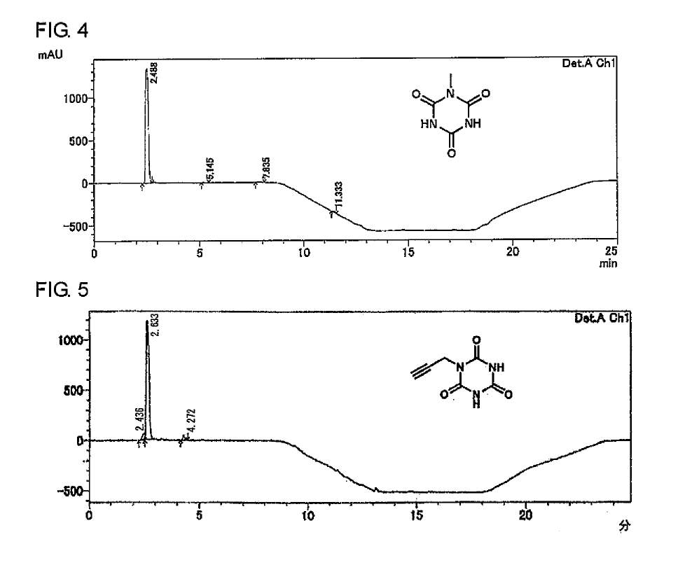

2.00 g of the monomethyl triazin-one compound obtained in Example 7, 20.00 g of 1,4-dioxane, and 0.48 g of methanol were mixed. To the mixture, 0.93 g of trifluoromethanesulfonic acid (available from Tokyo Chemical Industry Co., Ltd.) was added dropwise with stirring at 25° C. After completion of dropwise addition, the mixture was stirred at 25° C. for 2 hours to obtain a reaction solution. To the reaction solution, 0.63 g of triethylamine was added. The solvent was distilled off under reduced pressure, and the residue was then dried at 40° C. under reduced pressure. To the residue, 6.00 g of ethyl acetate was then added, and the mixture was stirred at 25° C. for 10 minutes. After stirring, the mixture was subjected to filtration, and the cake was washed with 2.00 g of ethyl acetate twice. The resulting crystal was dried at 40° C. under reduced pressure, to obtain 0.81 g of monomethylisocyanuric acid of formula (4a) described above as a white solid (yield: 91.0%). The obtained compound was subjected to measurement under the HPLC analysis condition-2. The purity thereof was 98.5%. This compound was subjected to ¹H NMR measurement (500 MHz, DMSO-d₆). A measurement result of ¹H NMR: δ 11.39 (s, 2H), 3.05 (s, 3H). A chromatogram obtained by the measurement is shown in FIG. 4.

### Example 10

0.81 g of the monomethylisocyanuric acid of formula (4a) was obtained as a white solid (yield: 91.4%) in the same manner as in Example 9 except that chloroform was used as a washing solvent. The obtained compound was subjected to measurement under the HPLC analysis condition-2. The purity thereof was 98.2%.

### Example 11

0.67 g of the monomethylisocyanuric acid of formula (4a) was obtained as a white solid (yield: 75.6%) in the same manner as in Example 9 except that ethanol was used as a washing solvent. The obtained compound was subjected to measurement under the HPLC analysis condition-2. The purity thereof was 98.2%.

### Comparative Example 13

0.91 g of the monomethylisocyanuric acid of formula (4a) was obtained as a white solid (yield: 102.1%) in the same manner as in Example 9 except that toluene was used as a washing solvent. The obtained compound was subjected to measurement under the HPLC analysis condition-2. The purity thereof was 97.2%.

### Comparative Example 14

1.68 g of the monomethylisocyanuric acid of formula (4a) was obtained as a white solid (yield: 189.1%) in the same manner as in Example 9 except that heptane was used as a washing solvent. The obtained compound was subjected to measurement under the HPLC analysis condition-2. The purity thereof was 95.6%.

### Comparative Example 15

0.92 g of the monomethylisocyanuric acid of formula (4a) was obtained as a white solid (yield: 103.2%) in the same manner as in Example 9 except that cyclopentyl methyl ether was used as a washing solvent. The obtained compound was subjected to measurement under the HPLC analysis condition-2. The purity thereof was 96.3%.

TABLE 4

| | | Yield [%] | Purity [%] |
|---|---|---|---|
| Example 9 | Ethyl acetate | 91.0 | 98.5 |
| Example 10 | Chloroform | 91.4 | 98.2 |
| Example 11 | Ethanol | 75.6 | 98.2 |
| Comparative | Toluene | 102.1 | 97.2 |
| Example 13 | | | |
| Comparative | Heptane | 189.1 | 95.6 |
| Example 14 | | | |
| Comparative | Cyclopentyl methyl ether | 103.2 | 96.3 |
| Example 15 | | | |

The results in Examples 9 to 11 show that an isocyanuric acid derivative having a hydrocarbon group can be obtained with high purity (98% or more). A method for producing such an isocyanuric acid derivative does not include a step that is carried out at a temperature of higher than 100° C. and a purification step by column chromatography, and thus the method is industrially useful.

### Comparative Example 16

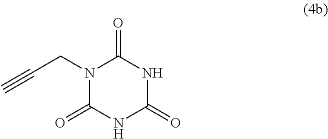

100.00 g of the triazin-one compound obtained in Example 4, 131.67 g of cesium carbonate (available from Tokyo Chemical Industry Co., Ltd.), and 1000.0 g of N-methyl-2-pyrrolidone were mixed, and the mixture was stirred at 40° C. for 30 minutes. The mixture was then cooled to lower than 5° C. To the mixture, 48.07 g of propargyl bromide (available from Tokyo Chemical Industry Co., Ltd.) was added dropwise. After completion of dropwise addition, the mixture was stirred at lower than 5° C. for 6 hours to obtain a reaction solution. To the reaction solution, 1000.0 g of toluene was added, and the mixture was subjected to filtration. To the filtrate, 1000.0 g of water was added, and separation was carried out. This operation was repeated three times. To this solution, 14.55 g of trifluoromethanesulfonic acid (available from Tokyo Chemical Industry Co., Ltd.) and 24.86 g of methanol were added dropwise. After completion of dropwise addition, the mixture was stirred at 25° C. for 2 hours to obtain a reaction solution. To the reaction solution, 39.26 g of triethylamine was added. The solvent was distilled off under reduced pressure, and the residue was then dried at 40° C. under reduced pressure. To the residue, 300.00 g of ethyl acetate was then added, and the mixture was stirred at 25° C. for 10 minutes. After stirring, the mixture was subjected to filtration, and the cake was washed with 100.00 g of ethyl acetate twice. The resulting crystal was dried at 40° C. under reduced pressure, to obtain 14.26 g of monopropargylisocyanuric acid of formula (4b) described above as a white solid (yield: 26.4%). The obtained compound was subjected to measurement under the HPLC analysis condition-2. The purity thereof was 94.0%. This compound was subjected to ¹H NMR measurement (500 MHz, DMSO-d₆). A measurement result of ¹H NMR: δ 11.56 (s, 2H), 4.39 (d, 2H), 3.20 (t, 1H). A chromatogram obtained by the measurement is shown in FIG. 5. Comparative Example 16 is an example in which a solution containing the compound of formula (3) that is obtained without washing with at least one solvent selected from the group consisting of alcohols and ethers is applied to the fourth step.

### INDUSTRIAL APPLICABILITY

For example, an isocyanuric acid derivative having a hydrocarbon group produced by the present invention can be applied to an anti-reflective coating-forming composition for lithography, a resist underlayer film-forming composition, a resist upper layer film-forming composition, a photocurable resin composition, a thermosetting resin composition, a flattened film-forming composition, an adhesive composition, and other compositions.

1. A method for producing an isocyanuric acid derivative having one hydrocarbon group comprising:
a first step of washing a reaction product obtained by reacting a compound of the following formula (0) with benzyl alcohol in which at least one hydrogen atom in a benzene ring is optionally substituted with methyl group in the presence of a base selected from the group consisting of a tertiary amine, an alkali metal carbonate, and an alkali metal hydrogencarbonate, with alcohols to obtain a compound of the following formula (1);
a second step of washing a reaction product obtained by reacting the compound of formula (1) in a solution containing a heterocyclic compound selected from the group consisting of N-methylmorpholine, N-ethylmorpholine, N-methylpiperidine, N-ethylpiperidine, N-methylpyrrolidine, and N-ethylpyrrolidine, and acetic acid or formic acid, with at least one solvent selected from the group consisting of esters, aromatic hydrocarbons, alcohols, and ethers to obtain a compound of the following formula (2);
a third step of washing a reaction product obtained by reacting the compound of formula (2) with a compound of the following formula (5), (6), (7), or (8) having a C₁₋₁₀ hydrocarbon group in the presence of alkali metal carbonate, with at least one solvent selected from the group consisting of alcohols and ethers to obtain a compound of the following formula (3); and
a fourth step of washing a reaction product obtained by reacting the compound of formula (3) with an alcohol compound in the presence of trifluoromethanesulfonic acid or trimethylsilyl trifluoromethanesulfonate, with at least one solvent selected from the group consisting of esters, halogenated alkyls, and alcohols to obtain a compound of the following formula (4): 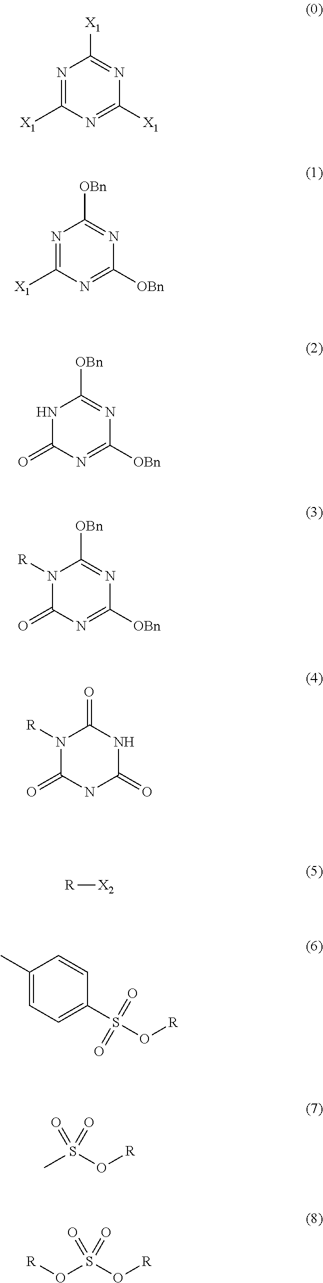
(wherein X₁s are each a chlorine atom, a fluorine atom, or a bromine atom, Bn is a benzyl group in which at least one hydrogen atom in a benzene ring is optionally substituted with methyl group, and R is a C₁₋₁₀ hydrocarbon group, and X₂ is a halogen atom), wherein all the steps are carried out at a temperature of not exceeding 100° C.

2. The method for producing an isocyanuric acid derivative having a hydrocarbon group according to claim 1, wherein the C₁₋₁₀ hydrocarbon group is an alkyl group.

3. The method for producing an isocyanuric acid derivative having a hydrocarbon group according to claim 1, wherein the benzyl alcohol and the base are each used in an amount of 2.7 molar equivalents to 5.0 molar equivalents relative to 1.0 molar equivalent of the compound of formula (0).

4. The method for producing an isocyanuric acid derivative having a hydrocarbon group according to claim 1, wherein the base is a tertiary amine.

5. The method for producing an isocyanuric acid derivative having a hydrocarbon group according to claim 1, wherein the alcohols used in the first step is ethanol.

6. The method for producing an isocyanuric acid derivative having a hydrocarbon group according to claim 1, wherein the at least one solvent used in the second step is selected from the group consisting of ethyl acetate, toluene, ethanol, and cyclopentyl methyl ether.

7. The method for producing an isocyanuric acid derivative having a hydrocarbon group according to claim 1, wherein the compound of formula (5), (6), (7), or (8) is an alkylating agent selected from the group consisting of halogenated alkyl, alkyl tosylate, alkyl mesylate, and dialkyl sulfate.

8. The method for producing an isocyanuric acid derivative having a hydrocarbon group according to claim 7, wherein the alkylating agent is methyl iodide, ethyl bromide, propyl bromide, allyl bromide, propargyl bromide, dimethyl sulfate, methyl p-toluenesulfonate, ethyl p-toluenesulfonate, or ethyl methanesufonate.

9. The method for producing an isocyanuric acid derivative having a hydrocarbon group according to claim 1, wherein the alkali metal carbonate used in the third step is potassium carbonate or cesium carbonate.

10. The method for producing an isocyanuric acid derivative having a hydrocarbon group according to claim 1, wherein the alcohols and ethers used in the third step are ethanol and cyclopentyl methyl ether.

11. The method for producing an isocyanuric acid derivative having a hydrocarbon groups according to claim 1, wherein the at least one solvent used in the fourth step is selected from the group consisting of ethyl acetate, chloroform, and ethanol.

12. The method for producing an isocyanuric acid derivative having a hydrocarbon group according to claim 1, wherein the temperature not exceeding 100° C. is 0° C. to 50° C.
